(19) United States
(12) Patent Application Publication
(10) Pub. No.: US 2026/0295164 A1
(43) Pub. Date: Oct. 1, 2026

(54) DRUG DELIVERY DEVICE

(71) Applicant: AMGEN INC., Thousand Oaks, CA (US)

(72) Inventors: Bjarke Lykke Ludvig SVENDSEN, Slagelse (DK); Christian PLAMBECH, Soeborg (DK); Matias MELANDER, Copenhagen (DK)

(73) Assignee: AMGEN INC., Thousand Oaks, CA (US)

(21) Appl. No.: 19/577,962

(22) Filed: Mar. 25, 2026

Related U.S. Application Data
(60) Provisional application No. 63/778,025, filed on Mar. 26, 2025.

(51) Int. Cl. A61M 5/20; A61M 5/315; A61M 5/32

(52) U.S. Cl. CPC A61M 5/2033; A61M 5/31578; A61M 5/3213; A61M 5/3245; A61M 2005/2013; A61M 2005/206; A61M 2005/208; A61M 2005/3254; A61M 2202/0007; A61M 2210/04

(57) ABSTRACT

A drug delivery device is provided comprising a housing having a first opening, a drug storage device including a delivery member having an insertion end configured to extend at least partially through the first opening in the housing, a plunger, a drive mechanism activatable to drive the plunger in a distal direction to expel a drug from the drug storage container, and a needle guard moveably disposed adjacent to the first opening. The needle guard has an extended position where the needle guard extends at least partially through the first opening and a retracted position where the needle guard is positioned away from the extended position. The needle guard has a base surface configured to contact an injection site and having an inner diameter defining a second opening and an outer diameter. A ratio of the outer diameter to the inner diameter is between about 3:1 and about 2:1.

### CROSS-REFERENCE TO RELATED APPLICATIONS

The present application claims priority to U.S. Provisional Patent Application No. 63/778,025, filed Mar. 26, 2025.

### FIELD OF DISCLOSURE

The present disclosure relates to drug delivery devices, and, more particularly, devices for automatically injecting a drug into a patient.

### BACKGROUND

A general aversion to exposed needles, as well as health and safety issues, have led to the development of drug delivery devices which conceal a needle or other insertion member prior to use and which automate various aspects of an injection process. Such devices offer a variety of benefits as compared with traditional methods of drug delivery including, for example, delivery via a conventional syringe.

Many drug delivery devices that conceal the insertion member prior to use and automate the injection process are actuated by pressing the device against the user's skin. However, depending on various user factors, pressing the device against the skin and exerting a force against the skin to activate the device may deform the underlying flesh and cause discomfort and, sometimes, even pain. Moreover, when pressing the device against the skin to activate the device, the needle shield may become enveloped by the underlying skin or tissue and prevent proper activation of the device. Furthermore, if the opening at the end of the needle shield is too large, the underlying skin or tissue may protrude into the opening and prevent accurate positioning and insertion of the insertion member at the injection site. Therefore, there is a need for an improved drug delivery device that causes the underlying skin at the injection site to be taut and firm. In addition, there is a need for an improved drug delivery device that increases the effective surface area at the injection site and distributes the activation force to an increased surface area to ensure accurate drug delivery while minimizing discomfort or pain.

The present disclosure sets forth drug delivery devices embodying advantageous alternatives to existing drug delivery devices, and that may address one or more of the challenges or needs mentioned herein.

### SUMMARY

One aspect of the present disclosure provides a drug delivery device comprising a housing having a first opening, a drug storage device including a delivery member having an insertion end configured to extend at least partially through the first opening in the housing, a plunger, a drive mechanism activatable to drive the plunger in a distal direction to expel a drug from the drug storage container through the delivery member, and a needle guard moveably disposed adjacent to the first opening. The needle guard has an extended position wherein the needle guard extends at least partially through the first opening and a retracted position wherein the needle guard is positioned away from the extended position toward the housing. The needle guard also has a base surface configured to contact an injection site, and the base surface has an inner diameter defining a second opening and an outer diameter. A ratio of the outer diameter to the inner diameter is between about 3:1 and about 2:1.

In some embodiments, the ratio of the outer diameter to the inner diameter may be between about 5:2 and about 2:1. In some embodiments, the outer diameter of the base surface may be between about 18 mm and about 25 mm, and the inner diameter of the base surface may be between about 9 mm and about 10 mm. In some embodiments, the needle guard may include a first portion at a proximal end thereof and a second portion having the base surface at a distal end thereof. At least the second portion may have a hollow and a cylindrical shape. In some embodiments, an inner diameter of the second portion may be equal to the inner diameter of the base surface. In some embodiments, an outer diameter of at least one of the first portion or the second portion may be equal to the outer diameter of the base surface. In other embodiments, an outer diameter of at least one of the first portion or the second portion may be less than the outer diameter of the base surface.

In some embodiments, the base surface may be a flat surface. In other embodiments, the base surface may be a curved surface. In some embodiments, in at least the extended position, the needle guard may be configured to surround the insertion end of the delivery member, and movement of the needle guard toward the retracted position may be configured to expose the insertion end of the delivery member. In some embodiments, the drug delivery device may further comprise a needle shield removably coupled to the insertion end of the delivery member, and an outer diameter of the needle shield may be less than the inner diameter of the base surface such that the needle shield can move through the second opening of the base surface.

Another aspect of the present disclosure provides a drug delivery device comprising a housing having a first opening, a drug storage device including a delivery member having an insertion end configured to extend at least partially through the first opening in the housing, a plunger, a drive mechanism activatable to drive the plunger in a distal direction to expel a drug from the drug storage container through the delivery member, and a needle guard moveably disposed adjacent to the first opening. The needle guard has an extended position wherein the needle guard extends at least partially through the first opening and a retracted position wherein the needle guard is positioned away from the extended position toward the housing. The needle guard also has a base surface having a second opening and configured to contact an injection site. The insertion end is configured to extend through the second opening when the needle guard is in the retracted position, and a surface area of the base surface is between about 200 mm² and about 315 mm².

In some embodiments, the surface area of the base surface may be between about 210 mm² and about 315 mm². In some embodiments, the base surface may have an inner diameter defining the second opening and an outer diameter. A ratio of the outer diameter to the inner diameter may be between about 3:1 and about 2:1.

In some embodiments, the needle guard may include a first portion at a proximal end thereof and a second portion having the base surface at a distal end thereof, and at least the second portion may have a hollow and cylindrical shape. In some embodiments, the base surface may be a flat surface. In other embodiments, the base surface may be a curved surface. In some embodiments, in at least the extended position, the needle guard may be configured to surround the insertion end of the delivery member, and movement of the needle guard toward the retracted position may be configured to expose the insertion end of the delivery member. In some embodiments, the drug delivery device may further comprise a rigid needle shield removably coupled to the insertion end of the delivery member, and an outer diameter of the rigid needle shield may be less than a diameter of the second opening such that the rigid needle shield can move through the second opening of the base surface.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description.

FIG. 1A is a cross-sectional view of an exemplary drug delivery device, in accordance with various embodiments of the present disclosure, in a pre-delivery state.

FIG. 1B is a cross-sectional view of the drug delivery device in FIG. 1A, in accordance with various embodiments of the present disclosure, in a delivery state.

FIG. 2A is a cross-sectional view of a section of an exemplary needle guard mechanism that illustrates an exemplary interference arrangement of the needle guard mechanism, in accordance with various embodiments of the present disclosure.

FIG. 2B is a perspective view of the needle guard mechanism in FIG. 2A, in accordance with various embodiments of the present disclosure.

FIG. 3 is a bottom, perspective view of the drug delivery device in FIG. 1A that illustrates an exemplary base surface of the needle guard mechanism, in accordance with various embodiments of the present disclosure.

### DETAILED DESCRIPTION

The present disclosure generally relates to drug delivery devices operable by a user for administering a drug, or in the case where a patient is the user, self-administering a drug. Various features are disclosed to distribute the activation force at the injection site and reduce or minimize any discomfort or pain during drug administration. Such features include, for example, an increased effective surface area at the distal end of the needle guard. The increased effective surface area at the distal end of the needle guard may cause the underlying skin at the injection site to be taut and firm and ensure proper injection and drug delivery. The increased effective surface area at the distal end of the needle guard may also prevent the needle guard from being enveloped by the underlying skin, which could prevent activation of the drug delivery device. These and other advantages will be apparent to one of ordinary skill in the art reviewing the present disclosure.

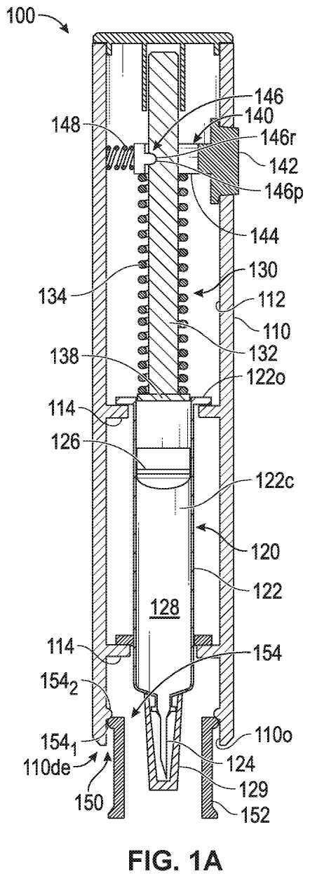

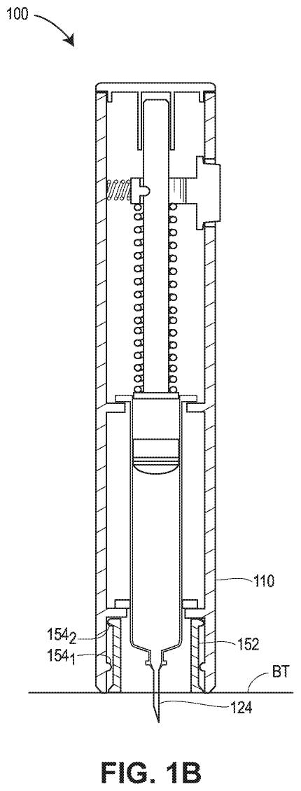

FIG. 1A illustrates a hand-held drug delivery device 100, in accordance with various embodiments of the present disclosure. The drug delivery device 100 comprises a needle guard mechanism 150 having a needle guard 152 that is movable or retractable to effect needle insertion. The drug delivery device 100 can be configured as a single-use, disposable injector or a multiple-use reusable injector. The drug delivery device 100 can be configured to deliver any suitable medicament or drug including those having a viscosity which can range, for example, from about 1-200 centipoise. Further, the drug delivery device 100 can be configured as an autoinjector for self-administration, although such devices can also be used by a caregiver or a formally trained healthcare provider to administer an injection.

The configuration of various components included in the drug delivery device 100 may depend on the operational state of the drug delivery device 100. The drug delivery device 100 may have a pre-delivery or storage state, a delivery or dosing state, and a post-delivery state, although fewer or more states are possible. The pre-delivery state may correspond to the configuration of the drug delivery device 100 subsequent to assembly and prior to activation by the user. In some embodiments, the pre-delivery state may exist in the time between when the drug delivery device 100 leaves a manufacturing facility and when a patient or user activates a drive mechanism of the drug delivery device 100. This includes the moments in time after the user has removed the drug delivery device 100 from any secondary packaging and prior to positioning the drug delivery device 100 against the injection site. The delivery state may correspond to the configuration of the drug delivery device 100 while drug delivery is in progress. The post-delivery state may correspond to the configuration of the drug delivery device 100 after drug delivery is complete and/or when a stopper is arranged in an end-of-dose position in a drug storage device.

Referring to FIG. 1A, the drug delivery device 100 in various embodiments can further comprise an elongated housing or outer casing 110 that holds a drug storage device 120. The drug storage device 120 in various embodiments can include a primary container 122, a stopper 126 moveably disposed in an interior chamber 122c of the primary container 122 for expelling a medicament or drug 128 contained therein, and an injection needle 124 (as shown), cannula or any other suitable dose delivery member or element capable of penetrating body tissue (BT) and dispensing a drug into the body of a patient. In some embodiments, the drug storage device 120 may comprise a conventional glass or plastic syringe or cartridge that is fillable with the one or more doses of the medicament or drug 128. In some embodiments, the drug storage device 120 may comprise a pre-filled syringe that is pre-filled with the one or more doses of the medicament or drug 128. A removable needle shield 129 can be installed over the injection needle 124 for maintaining same in a sterile state prior to use of the drug delivery device 100.

In various embodiments, the interior surface 112 of the housing 110 can include one or more support members 114 for holding the drug storage device 120 in a fixed manner within the outer housing 110 with at least an insertion end of the injection needle 124 extending through an opening 1100 defined in a distal end 110de of the housing 110.

Referring still to FIG. 1A, various embodiments of the drug delivery device 100 can further comprise an injection drive mechanism 130 and a drive triggering mechanism 140. The injection drive mechanism 130 can be disposed within the housing 110 and in some embodiments may comprise a plunger 132 and a plunger drive spring 134 for propelling the plunger 132 into and through the primary container 122 of the drug storage device 120 to perform drug injection or dosing. The plunger 132 and the plunger drive spring 134 can be configured so that the plunger 132 extends through the plunger drive spring 134 with one end of the spring 134 engaging a head member 138 of the plunger 132 and the other end of the spring 134 engaging the drive triggering mechanism 140. Prior to activation of the injection drive mechanism 130, in some embodiments the plunger 132 is in a position where the head member of the plunger 132 is disposed adjacent to the opening 1220 of the primary container 122 of the drug storage device 120 with the spring 134 compressed between the head member 138 of the plunger 132 and the drive triggering mechanism 140. When the injection drive mechanism 130 is activated by the drive triggering mechanism 140, the plunger drive spring 134 expands to propel the plunger 132 into and through the primary container 122 of the drug storage device 120 to drive the stopper 126 through the primary container 122 to expel the drug 128 through the injection needle 124.

In other embodiments, the injection drive mechanism 130 of the drug delivery device 100 can comprise an electrical/mechanical arrangement (not shown) comprising one or more motors and/or solenoids and a drive train or transmission, or an arrangement that generates or releases a pressurized gas or fluid (not shown), to propel the plunger 132. In further embodiments, the injection drive mechanism 130 may comprise a mechanical arrangement that generates or releases a pressurized gas or fluid (not shown) which acts directly on the stopper 126 to move it through the primary container 122 to expel the drug 128 therefrom through the injection needle 124.

Referring still to FIG. 1A, the drive triggering mechanism 140 can be disposed within the housing 110 and may comprise an activation button 142 which extends through an opening in the side of the housing 110, a plunger release arm 144 extending from the activation button 142, and a trigger spring 148 disposed between the plunger release arm 144 and the interior surface 112 of the housing 110. The activation button 142 enables the drive triggering mechanism 140 to be actuated by the patient or other operator, which in turn, activates the injection drive mechanism 130. The plunger release arm 144 is moveable between a plunger hold position and a plunger release position. In some embodiments, in the plunger hold position, the plunger release arm 144 holds the plunger 143 in position via a detent 146. The detent 146 may comprise a projection 146p on the plunger release arm 144 and a recess 146r formed in the side of the plunger 132. In the plunger release position, the plunger release arm 144 disengages the detent projection 146p from the detent recess 146r, which releases the plunger 132 and allows it to be propelled by the plunger drive spring 134. The trigger spring 148 applies a force which maintains the plunger release arm 144 in the plunger hold position with the detent projection 146p and recess 146r engaged. When the activation button 142 is pressed, the force of the trigger spring 148 is overcome and the plunger release arm 144 is moved out of the plunger hold position into the plunger release position, thereby disengaging the detent 146 and releasing the plunger 132. In other embodiments, the drive triggering mechanism 140 can comprise an electrical/mechanical arrangement (not shown) comprising one or more switches, springs, and/or sensors to activate the injection drive mechanism 130.

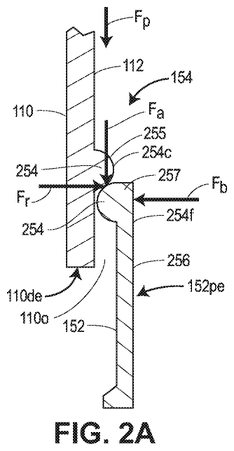

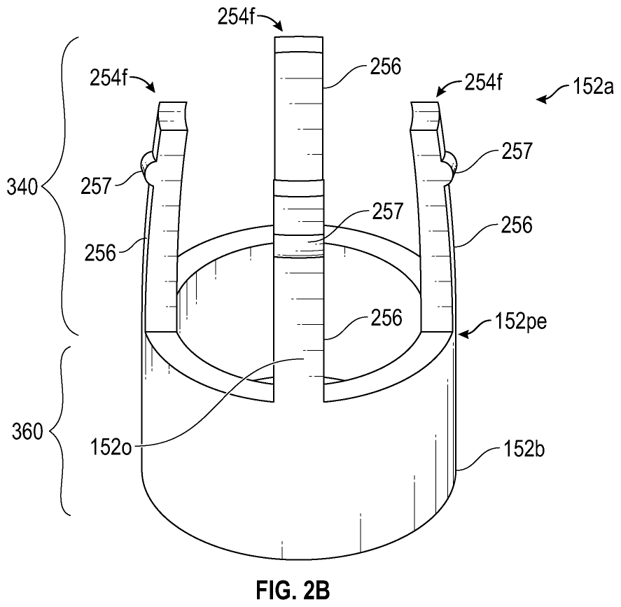

Referring still to FIG. 1A, the needle guard mechanism 150 in various embodiments comprises a needle guard 152 movably disposed at the distal end 110de of the housing 110 adjacent to the opening 1100. The needle guard 152 may have a hollow and generally cylindrical or tubular shape. The needle guard 152 may have a proximal end 152a received within the housing 110 and a distal end 152b, and may be configured to move relative to the housing 110 between an extended position (FIG. 1A) wherein the distal end 152b extends through the opening 1100 in the housing 110 and a retracted position (FIG. 1B) wherein the distal end 152b is retracted, fully or partially, into the opening 1100 in the housing 110. In at least the extended position, the needle guard 152 may extend beyond and surround the insertion end of the injection needle 124 extending from the distal end 110de of the housing 110 to protect against accidental needle sticks. The needle guard 152 can be configured so that it collapses or retracts in a proximal direction into the housing 110, as shown in FIG. 1B (retracted position). Moving the needle guard 152 toward the retracted position may expose the insertion end of the injection needle 124 such that at least the insertion end of the injection needle 124 extends through the opening 1100 in the housing 110. The proximal end 152a and the distal end 152b of the needle guard 152 may include, respectively, a first portion 340 (FIG. 2B) and a second portion 360 (FIG. 2B) configured to contact the body tissue (BT) at the injection site. At least the second portion 360 of the needle guard 152 may have a hollow and generally cylindrical or tubular shape. In some embodiments, the first portion 340 and the second portion 360 may define a single, monolithic structure. Said another way, the first portion 340 and the second portion 360 may be constructed in one piece. In other embodiments, the first portion 340 and the second portion 360 may be physically separate structures that are fixedly attached to each other such that they are immovable relative to each other and/or move jointly when in motion.

The needle guard mechanism 150 may also comprise a detent or interference arrangement 154. The needle guard 152 may be biased into an extended position depicted in FIG. 2A with a spring (not shown). The interference arrangement 154 retains the needle guard 152 in the extended position with a tunable or selectable threshold of resistance to the patient's or operator's effort to retract the needle guard 152 in a proximal direction relative to the housing 110 and insert the injection needle 124 of the drug storage device 120 into body tissue (BT) as shown in FIG. 1B, thereby optimizing needle insertion speed and force and preventing unintended dosing after the needle shield 129 has been removed.

The interference arrangement 154 in various embodiments can comprise a first member 1541 associated with the housing 110, and a second member 1542 associated with the needle guard 152. At least portions of the first and second members 1541, 1542 engage one another to retain the needle guard 152 in the extended position and provide the selected threshold of resistance to movement or retraction of the needle guard 152. In some embodiments, one of the first and second members 1541, 1542 can be configured to move when an axial force is applied to the drug delivery device 100 by the patient or operator pressing the device 100 toward the injection site meets or exceeds the selected threshold of resistance, thereby allowing the other one of the first and second members 1541, 1542 to slide past it to release the needle guard 152 and allow it to retract toward the housing 110 into the retracted position to achieve needle insertion, as shown in FIG. 1B.

Referring now to FIG. 2A, in some embodiments, the interference arrangement 154 can comprise an overcenter cam mechanism 254 comprising a fixed cam element 254c and one or more resiliently biased cam followers 254f. In some embodiments, the fixed cam element 254c can comprise a circumferential protrusion 255 disposed on the interior surface 112 of the housing 110 adjacent to the opening 1100 at the distal end 110de of the housing 110. The circumferential protrusion can be continuous or segmented. As shown in FIG. 2B, at least the first portion 340 of the needle guard 152 may comprise the resiliently biased cam followers 254f. In some embodiments, the one or more of the resiliently biased cam followers 254f can comprise a flexible arm 256 that extends from a proximal end 152pe of the needle guard 152 and a projection 257 disposed on an outwardly facing surface 1520 of the flexible arm 256. As shown in FIG. 2A, the one or more flexible arms 256 bias their corresponding projections 257 against the fixed circumferential protrusion 255.

As also illustrated in FIG. 2A, the fixed cam element 254c on the casing 110 and the cam followers 254f on the needle guard 152 include rounded (e.g., cammed or bulbous) external surfaces (e.g., profiles) to facilitate sliding interaction during use of the drug delivery device 100. For example, as a user applies an axial force Fp to the drug delivery device 100 against an injection site, a generally equal and opposite axial force is applied to the needle guard 152 urging the needle guard 152 in the proximal direction toward its retracted position. But, the engagement of the cam element 254c and cam followers 254f prevents substantial retraction until the threshold of resistance is overcome. That is, in the version of FIG. 2A, the threshold of resistance is equal to the force required to cause the flexible arms 256 carrying the cam followers 254f to deflect away from the fixed cam element 254 such that the projections 257 of the cam followers 254f are able slide in a proximal direction relative to and beyond the fixed cam element 254c. In FIG. 2A, the force applied by the user (also referred to as “activation force”) is shown as Fp, and this translates into the fixed cam element 254c applying an axial force component Fa and a radial force component Fr on each of the cam followers 254f. Thus, in order to overcome the threshold of resistance, the axial force Fp applied by the user must be large enough to create a radial force component Fr that is sufficient to overcome a natural resistive bias force Fb of the flexible arms 256. The resistive bias force Fb of the flexible arms 256 is primarily dependent on the material from which the arms 256 are constructed and the geometry of the arms 256 (e.g., the length of the arms 256, the width of the arms 256, the thickness of the arms 256, a cross-sectional shape of the arms, etc.).

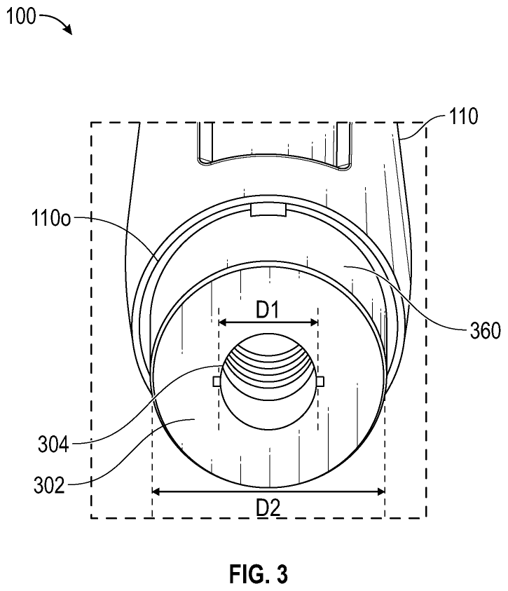

Referring now to FIG. 3, the second portion 360 of the needle guard 152 may include a base surface 302 at a distal end thereof, and the base surface 302 may be configured to abut and contact the body tissue (BT) at the injection site during operation of the drug delivery device 100. In accordance with the embodiments of the present disclosure, the base surface 302 may be generally circular in shape. The generally circular shape of the base surface 302 may ensure even distribution of the activation force and/or the holding force around the body tissue at the injection site.

The base surface 302 may have an inner diameter D1 defining an opening 304 and an outer diameter D2. In some embodiments, the outer diameter D2 of the base surface 302 may be between about 15 mm and about 30 mm. For example, in some embodiments, the outer diameter D2 may be about 18 mm, 19 mm, 20 mm, 21 mm, 22 mm, 23 mm, or 25 mm. In some embodiments, the outer diameter D2 may be about 19 mm, 20 mm, 21 mm, or 22 mm. The inner diameter D1 of the base surface 302 may be between about 9 mm and about 11 mm. For example, in some embodiments, the inner diameter D1 may be between about 9 mm and about 10 mm. In some embodiments, the inner diameter D1 may be about 9.2 mm, 9.3 mm, 9.4 mm, 9.5 mm, or 9.6 mm. The inner diameter D1 and the outer diameter D2 of the base surface 302 may be selected such that the ratio of the outer diameter D2 to the inner diameter D1 is between about 3:1 and about 2:1. In some embodiments, the inner diameter D1 and the outer diameter D2 of the base surface 102 may be selected such that the ratio of the outer diameter D2 to the inner diameter D1 is between about 5:2 and about 2:1. In some embodiments, the surface area of the base surface 302 configured to be in contact with the body tissue may be between about 200 mm² and about 315 mm². For example, in some embodiments, the surface area may be between about 210 mm² and about 315 mm².

In the present embodiment, the inner diameter D1 of the base surface 302 is between about 9.3 mm and about 9.4 mm, and the outer diameter D2 of the base surface 302 is between about 19 mm and about 22 mm. Accordingly, the ratio of the outer diameter D2 to the inner diameter D1 is between about 5:2 and about 2:1. Moreover, in the present embodiment, the surface area of the base surface 302 is between about 210 mm² and about 315 mm². The inventors of the present disclosure have found that many conventional drug delivery devices, and particularly autoinjectors, have needle guards with similar inner and outer diameters, thereby resulting in a ratio of outer diameter to inner diameter of approximately 3:2 or less. Because the outer diameter of the needle guard is only slightly larger than the inner diameter in many conventional devices, the surface area of the base surface of the needle guard contacting the patient's skin is small. The inventors of the present disclosure have found that having a small surface area at the base of the needle guard may be undesirable and/or problematic for various reasons. For instance, when depressing the needle guard against the patient's skin to activate the drug delivery device, the needle guard may become enveloped by the underlying skin or body tissue due to the small surface area at the base of the needle guard, and thereby prevent activation of the device. Moreover, when depressing the needle guard against the patient's skin, the underlying skin or tissue may bulge into the opening, such as the opening 304 at the base surface 302 (shown in FIG. 3), due to the small surface area at the base of the needle guard, and thereby inhibit accurate positioning and insertion of the needle at the injection site. Furthermore, when the user exerts a force on the device to activate the device and hold the device during drug delivery, the small surface area at the base of the needle guard may increase the pressure against the patient's skin and cause discomfort and even pain.

For example, the maximum pressure exerted against the patient's skin at the injection site may be calculated using the following equation:

$${P}_{max} = \frac{{F}_{max}}{{A}_{base}}$$

where Pₘₐₓ is the maximum pressure exerted against the patient's skin, Fₘₐₓ is the maximum force applied by the user when activating the drug delivery device, and A_{base} is the surface area at the base surface of the needle guard configured to be in contact with the patient's skin.

The inventors have found that maintaining the maximum pressure (Pₘₐₓ) at the injection site below about 0.05 N/mm² during activation of the drug delivery device advantageously helps prevent discomfort and pain for the user. To maintain such pressure, the inventors have found that, based on the average force (Fₘₐₓ) applied by the user when activating an autoinjector by depressing the needle guard 152 against the body tissue (BT), it is advantageous for the surface area at the base surface 302 (A_{base}) to exceed about 200 mm² to reduce deformation of the underlying skin or tissue and prevent discomfort and pain. FIG. 3 illustrates an exemplary base surface 302 of the needle guard 152 of the drug delivery device 100 that is constructed in accordance with these principles found by the inventors of the present disclosure. Accordingly, the inventors of the present disclosure have advantageously chosen the inner diameter D1 and the outer diameter D2 of the base surface 302 of the needle guard 152 (and accordingly, the resultant, specific range of ratio of outer diameter to inner diameter) to: (a) reduce deformation of the underlying body tissue, (b) prevent discomfort and pain at the injection site, (c) prevent bulging of the body tissue into the opening 304, (d) prevent the needle guard 152 (and particularly the second portion 360) from being enveloped by the body tissue, and (e) provide the base surface 302 with an increased effective surface area at the injection site to keep the underlying body tissue taut and firm and improve the distribution of the activation force and/or holding force at the injection site.

In some embodiments, as shown in FIG. 3, the base surface 302 may be a flat surface. In other embodiments, the base surface 302 may be a curved surface, such as a convex surface. The flat and/or convex surface of the base surface 302 may, for example, prevent bulging of the body tissue into the opening 304 and, thus, ensure accurate positioning and insertion of the injection needle 124 at the injection site. Moreover, the flat and/or convex surface of the base surface 302 may keep the body tissue at the injection site taut and firm to further ensure proper injection and drug delivery. In some embodiments, as shown in FIG. 2B, at least the second portion 360 of the needle guard 152 may have a hollow and generally cylindrical shape. In some embodiments, both the first portion 340 and the second portion 360 of the needle guard 152 may have a hollow and generally cylindrical shape. In some embodiments, the inner diameter of the second portion 360 may be equal to the inner diameter D1 of the base surface 302. In other words, the opening 304 (FIG. 3) at the base surface 302 may extend proximally into the second portion 360 of the needle guard 152. In some embodiments, the outer diameter of the first portion 340 and/or the second portion 360 of the needle guard 152 may be equal to the outer diameter of the base surface 302 such that the needle guard 152 has a cylindrical shape. In other embodiments, the outer diameter of at least one of the first portion 340 or the second portion 360 may be less than the outer diameter of the base surface 302 such that the base surface 302 flares radially outward from at least one of the first portion 340 or the second portion 360.

In some embodiments, the outer diameter of the needle shield 129 may be less than the inner diameter D1 of the base surface 302 such that the needle shield 129 can be movably accommodated within the opening 304 and can move through the opening 304 of the base surface 302 to remove the needle shield 129 prior to drug delivery. In other words, the inner diameter D1 of the base surface 302 is advantageously chosen such that the needle shield 129 may be movably disposed within the opening 304 at the base surface 302 of the needle guard 152.

Having described the general configuration and operation of the drug delivery device 100, a method of using the drug delivery device 100 to perform an injection will now be described with reference to FIGS. 1A, 1B, 2A, 2B, and 3. As a preliminary step, the user may remove the drug delivery device 100 from any secondary packaging such as a plastic bag and/or cardboard box. Also, as a preliminary step, the user may prepare the injection site, e.g., by rubbing the body tissue with an alcohol wipe. Next, the user may pull and detach a removable cap (not shown) from the housing 110. In some embodiments, the removable cap may comprise a gripper (not shown) that is configured to engage the needle shield 129. As a result of pulling and detaching the removable cap, the gripper may pull and detach the needle shield 129 from the primary container 122. The needle shield 129 may be removed through the opening 304 at the base surface 302 of the needle guard 152. This may uncover the insertion end of the injection needle 124. Nevertheless, the insertion end of the injection needle 124 will remain surrounded by the needle guard 152 at this stage, as shown in FIG. 1A. Next, the user may position the second portion 360 of the needle guard 152 over the body tissue such that the base surface 302 is abutting the body tissue and, then, push the base surface 302 against the body tissue at the injection site. The activation force (Fp) applied by the user must be large enough to create a radial force component Fr that is sufficient to overcome a natural resistive bias force Fb of the flexible arms 256 and cause the needle guard 152 to retract into the opening 1100 of the housing 110. The injection needle 124 remains stationary relative to the housing 110 during the retracting movement of the needle guard 152.

As discussed above, when applying the activation force to cause the needle guard 152 to retract, the drug delivery device 100 is able to reduce deformation of the underlying body tissue, prevent discomfort and pain at the injection site, prevent bulging of the body tissue into the opening 302, and prevent the needle guard 152 from being enveloped by the body tissue due to the inner diameter D1, the outer diameter D2, the specific range of ratio of the outer diameter D2 to the inner diameter D1, and the resulting surface area of the base surface 302 advantageously chosen by the inventors of the present disclosure. Moreover, the base surface 302 with an increased effective surface area at the injection site may advantageously keep the body tissue taut and firm and improve the distribution of the activation force and/or holding force at the injection site. Furthermore, the shape of the base surface 302 (e.g., generally circular shape, flat and/or convex surface) may advantageously keep the body tissue taut and firm and ensure even distribution of the activation force and/or holding force at the injection site.

Once the needle guard 152 is retracted, the drive triggering mechanism 140 may be actuated to activate the injection drive mechanism 130. During activation of the injection drive mechanism 130, the plunger release arm 144 may move from the plunger hold position to the plunger release position to disengage the detent projection 146p from the detent recess 146r. This releases the plunger 132 and allows it to be propelled by the plunger drive spring 134 to expel the drug 128 from the primary container 122 of the drug storage device 120 through the injection needle 124.

From the foregoing, it can be seen that the present disclosure advantageously provides a streamlined design for a drug delivery device having automated features. Various mechanisms and components of the drug delivery device may interact with each other in synergistic ways so as to limit the number of moving parts required by the drug delivery device, thereby improving the reliability of the drug delivery device and saving costs, as well as providing other benefits and advantages.

As will be recognized, the devices and methods according to the present disclosure may have one or more advantages relative to conventional technology, any one or more of which may be present in a particular embodiment in accordance with the features of the present disclosure included in that embodiment. Other advantages not specifically listed herein may also be recognized as well.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP Ilb/llia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFß mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity® (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. In some embodiments, the drug delivery device may contain or be used with Bemarituzumab, a monoclonal antibody that inhibits fibroblast growth factor receptor 2b (FGFR2b) for the treatment of advanced Gastric and Gastroesophageal Junction (GEJ) cancers. Additionally, bispecific T cell engager (BiTE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with ParsabivT® (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 206 (nivolumab), a biosimilar candidate to OPDIVO®, or another product containing a monoclonal antibody that targets the PD-1 protein on T cells. In some embodiments, the drug delivery device may contain or be used with ABP 234 (pembrolizumab), a biosimilar candidate to KEYTRUDA®, or another product containing a monoclonal antibody that binds to the PD-1 protein on immune cells. In some embodiments, the drug delivery device may contain or be used with ABP 692 (ocrelizumab), a biosimilar candidate to OCREVUS®, or another product containing a humanized anti-CD20 monoclonal antibody. In some embodiments, the drug delivery device may contain or be used with daxdilimab, a human monoclonal antibody against ILT7 or another product. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with Pavblu®, a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Imdelltra® (tarlatamab-dle), an anti-delta-like ligand 3 (DLL3) x anti-CD3 bispecific T cell engager (BiTE) molecule, or another product containing tarlatamab-dlle for the treatment of small cell lung cancer. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Lumakras® (sotorasib), a KRAS^{G12C} small molecule inhibitor, or another product containing a KRAS^{G12C} small molecule inhibitor. In some embodiments, the drug delivery device may contain or be used with Tezaspire® (tezepelumab-ekko), a human monoclonal antibody that inhibits the action of thymic stromal lymphopoietin (TSLP), or another product containing a human monoclonal antibody that inhibits the action of TSLP. In some embodiments, the drug delivery device may contain or be used with Tavneos® (avacopan), a complement 5a receptor 1 (C5aR1) antagonist that inhibits the effects of C5a. In some embodiments, the drug delivery device can contain or be used with Tepezza® (teprotumumab-trbw), a human monoclonal antibody against insulin-like growth factor-1 receptor (IGF-1R), or another product containing a human monoclonal antibody against IGF-1R. In some embodiments, the drug delivery device can contain or be used with Uplizna® (inebilizumab-cdon), a humanized monoclonal antibody that binds to the B cell-specific surface antigen CD19, or another product containing a humanized monoclonal antibody that binds to the B cell-specific surface antigen CD19. In some embodiments, the drug delivery device may contain or be used with rocatinlimab (AMG 451), a human anti-OX40 monoclonal antibody that is expressed on activated T cells and blocks OX40 to inhibit and/or reduce the number of OX40 pathogenic T cells that are responsible for driving system and local atopic dermatitis inflammatory responses. In some embodiments, the drug delivery device may contain or be used with ordesekimab (AMG 714), a human monoclonal antibody that binds to Interleukin-15 (IL-15) or another product containing a human monoclonal antibody that binds to Interleukin-15 (IL-15). In some embodiments, the drug delivery device may contain or be used with olpasiran (AMG 890), a small interfering RNA (siRNA) that lowers lipoprotein(a), also known as Lp(a), or another product containing a small interfering RNA (siRNA) that lowers lipoprotein(a). In some embodiments, the drug delivery device may contain or be used with Wezlana®/Wezenla™ (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 104, or another product containing a human anti-TSLP Fab. In some embodiments, the drug delivery device may contain or be used with AMG 193, or another product containing a small molecule methylthioadenosine (MTA) cooperative protein arginine methyltransferase 5 (PRMT5) inhibitor. In some embodiments, the drug delivery device may contain or be used with AMG 329, or another product containing a human monoclonal antibody that binds and neutralizes the function of the FLT3-ligand. In some embodiments, the drug delivery device may contain or be used with AMG 732, or another product containing a monoclonal antibody against insulin-like growth factor-1 receptor (IGF-1R). In some embodiments, the drug delivery device may contain or be used with AMG 305, or another product containing dual-targeting bispecific T cell engager (BiTE) molecule against P-cadherin (CDH3), mesothelin (MSLN) and CD3. In some embodiments, the drug delivery device may contain or be used with AMG 355, or another product containing an anti-CCR8 monoclonal antibody. In some embodiments, the drug delivery device may contain or be used with AMG 378, or another product containing a small molecule for the treatment of ulcerative colitis. In some embodiments, the drug delivery device may contain or be used with AMG 410, or another product containing a small molecule for the treatment of solid tumors. In some embodiments, the drug delivery device may contain or be used with AMG 513, or another product containing a molecule for the treatment of obesity. In some embodiments, the drug delivery device may contain or be used with AMG 691, or another product containing a monoclonal antibody for the treatment of asthma. In some embodiments, the drug delivery device may contain or be used with AMG 160, or another product that contains a half-life extended (HLE) anti-prostate-specific membrane antigen (PSMA) x anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with MariTide™ (AMG 133), or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1 x IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33 x anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3) x anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP x 4-1BB-targeting DARPin® biologic under investigation as a treatment for solid tumors. In some embodiments, the drug delivery device may contain or be used with AMG 509 (xaluritamig) or another product containing a bivalent T-cell engager and is designed using XmAb® 2+1 technology. In some embodiments, the drug delivery device may contain or be used with AMG 562 or another product containing a half-life extended (HLE) CD19 x CD3 BITE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3 x epidermal growth factor receptor vIII (EGFRVIII) BiTE® (bispecific T cell engager) molecule. In some embodiments, the drug delivery device may contain or be used with AMG 673 or another product containing a half-life extended (HLE) anti-CD33 x anti-CD3 BiTE® (bispecific T cell engager) construct. In some embodiments, the drug delivery device may contain or be used with AMG 701 or another product containing a half-life extended (HLE) anti-B-cell maturation antigen (BCMA) x anti-CD3 BiTE® (bispecific T cell engager) construct. In some embodiments, the drug delivery device may contain or be used with AMG 757 or another product containing a half-life extended (HLE) anti-delta-like ligand 3 (DLL3) x anti-CD3 BiTE® (bispecific T cell engager) construct. In some embodiments, the drug delivery device may contain or be used with AMG 910 or another product containing a half-life extended (HLE) epithelial cell tight junction protein claudin 18.2 x CD3 BiTE® (bispecific T cell engager) construct. In some embodiments, the drug delivery device may contain or be used with Dazodalibep, a fusion protein binding CD40L on T cells to block their interaction with CD40-expressing B cells.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above-described embodiments without departing from the spirit and scope of the invention(s) disclosed herein, and that such modifications, alterations, and combinations are to be viewed as being within the ambit of the inventive concept(s).

What is claimed is:

1. A drug delivery device comprising:
a housing having a first opening;
a drug storage device including a delivery member having an insertion end configured to extend at least partially through the first opening in the housing;
a plunger;
a drive mechanism activatable to drive the plunger in a distal direction to expel a drug from the drug storage container through the delivery member; and
a needle guard moveably disposed adjacent to the first opening, the needle guard having an extended position wherein the needle guard extends at least partially through the first opening and a retracted position wherein the needle guard is positioned away from the extended position toward the housing,
wherein the needle guard has a base surface configured to contact an injection site, the base surface having an inner diameter defining a second opening and an outer diameter, and
wherein a ratio of the outer diameter to the inner diameter is between about 3:1 and about 2:1.

2. The drug delivery device of claim 1, wherein the ratio of the outer diameter to the inner diameter is between about 5:2 and about 2:1.

3. The drug delivery device of claim 1, wherein the outer diameter of the base surface is between about 18 mm and about 25 mm, and wherein the inner diameter of the base surface is between about 9 mm and about 10 mm.

4. The drug delivery device of claim 1, wherein the needle guard includes a first portion at a proximal end thereof and a second portion having the base surface at a distal end thereof, wherein at least the second portion has a hollow and cylindrical shape.

5. The drug delivery device of claim 4, wherein an inner diameter of the second portion is equal to the inner diameter of the base surface.

6. The drug delivery device of claim 5, wherein an outer diameter of at least one of the first portion or the second portion is equal to the outer diameter of the base surface.

7. The drug delivery device of claim 5, wherein an outer diameter of at least one of the first portion or the second portion is less than the outer diameter of the base surface.

8. The drug delivery device of claim 1, wherein the base surface is a flat surface.

9. The drug delivery device of claim 1, wherein the base surface is a curved surface.

10. The drug delivery device of claim 1, wherein, in at least the extended position, the needle guard is configured to surround the insertion end of the delivery member, and wherein movement of the needle guard toward the retracted position is configured to expose the insertion end of the delivery member.

11. The drug delivery device of claim 1, further comprising a needle shield removably coupled to the insertion end of the delivery member, wherein an outer diameter of the needle shield is less than the inner diameter of the base surface such that the needle shield can move through the second opening of the base surface.

12. A drug delivery device comprising:
a housing having a first opening;
a drug storage device including a delivery member having an insertion end configured to extend at least partially through the first opening in the housing;
a plunger;
a drive mechanism activatable to drive the plunger in a distal direction to expel a drug from the drug storage container through the delivery member; and
a needle guard moveably disposed adjacent to the first opening, the needle guard having an extended position wherein the needle guard extends at least partially through the first opening and a retracted position wherein the needle guard is positioned away from the extended position toward the housing,
wherein the needle guard has a base surface having a second opening and configured to contact an injection site, wherein the insertion end is configured to extend through the second opening when the needle guard is in the retracted position, and wherein a surface area of the base surface is between about 200 mm² and about 315 mm².

13. The drug delivery device of claim 12, wherein the surface area of the base surface is between about 210 mm² and about 315 mm².

14. The drug delivery device of claim 12, wherein the base surface has an inner diameter defining the second opening and an outer diameter, and wherein a ratio of the outer diameter to the inner diameter is between about 3:1 and about 2:1.

15. The drug delivery device of claim 12, wherein the needle guard includes a first portion at a proximal end thereof and a second portion having the base surface at a distal end thereof, wherein at least the second portion has a hollow and cylindrical shape.

16. The drug delivery device of claim 12, wherein the base surface is a flat surface.

17. The drug delivery device of claim 12, wherein the base surface is a curved surface.

18. The drug delivery device of claim 12, wherein, in at least the extended position, the needle guard is configured to surround the insertion end of the delivery member, and wherein movement of the needle guard toward the retracted position is configured to expose the insertion end of the delivery member.

19. The drug delivery device of claim 12, further comprising a needle shield removably coupled to the insertion end of the delivery member, wherein an outer diameter of the needle shield is less than a diameter of the second opening such that the needle shield can move through the second opening of the base surface.
